(19) United States
(12) Patent Application Publication
(10) Pub. No.: US 2011/0236957 A1
(43) Pub. Date: Sep. 29, 2011

(54) Nanoparticle Labeling Reagents and Methods of Use

(75) Inventor: Kevin C. Weng, San Jose, CA (US)

(73) Assignee: THE REGENTS OF THE UNIVERSITY OF CALIFORNIA, Oakland, CA (US)

(21) Appl. No.: 13/122,397

(22) PCT Filed: Oct. 2, 2009
(86) PCT No.: PCT/US09/59394

§ 371 Date: Jun. 13, 2011

Related U.S. Application Data
(60) Provisional application No. 61/195,176, filed on Oct. 2, 2008.

(51) Int. Cl. C12N 7/00; C07K 16/44; C07H 21/00; C12N 5/071; C07C 237/10; C07C 43/13; G01N 33/53; C09K 11/06; B82Y 15/00; B82Y 5/00

(52) U.S. Cl. 435/235.1; 530/391.3; 536/23.1; 435/325; 564/153; 568/623; 436/501; 252/301.16; 977/773; 977/902; 977/834; 977/807; 977/797; 977/774; 977/762

(57) ABSTRACT

Compositions and methods for labeling biological targets using a conjugate of a luminescent component and a targeting molecule attached to a nanoparticle core structure are described. The labeling conjugates offer high intensity and low background, and are ideal for histology and pathology.

### CROSS-REFERENCE TO RELATED APPLICATIONS

This application claims the benefit of U.S. Provisional Application No. 61/195,176, filed Oct. 2, 2008, incorporated herein by reference in its entirety.

### TECHNICAL FIELD

The present compositions and methods relate to labeling cells and other biological materials using a single labeling reagent based on a nanoparticle core structure having a plurality of targeting molecules and/or luminescent nanocrystals, for multivalent presentation of the targeting molecules and/or nanocrystals.

### BACKGROUND

Various assays for use in detecting the presence or amount of a biological target are known in the art, Such assays typically include a targeting molecule that interacts with a preselected biological target, which interaction is detectable upon exposure to additional reagents. Exemplary types of biological assays are described, below:

Immunohistochemistry (IHC) refers to the process of localizing proteins or other targeting molecules that recognize cellular targets in cells or a tissue section. A typical immunohistochemistry (IHC) assay utilizes a “primary” antibody that binds specifically to the biological target, and a “secondary” antibody conjugated to an enzyme to detect the presence of the primary antibody. In the presence of a suitable substrate, the enzyme is capable of producing a colorimetric, fluorescent, or other detectable change in the substrate, thereby indirectly indicating the presence of the biological target.

Fluorescent in situ hybridization (FISH) refers to a cytogenetic technique used to detect and localize specific DNA sequences on chromosomes using fluorescently labeled nucleic acid probes. Fluorescence microscopy can be used to image bound fluorescent probes. FISH is often used for finding specific features in DNA that can be used in genetic counseling, medicine, and species identification.

Flow cytometry or fluorescence-activated cell sorting (FACS) refers to a technique for counting, examining, and sorting microscopic particles, typically cells, suspended in a stream of fluid (e.g., blood, saline, buffers, and the like). FACS allows simultaneous multiparametric analysis of the physical and/or chemical characteristics of single cells flowing through an optical and/or electronic detection apparatus. FACS is particularly useful for analyzing and quantifying biomarkers expressed on or in cells, which can be important in diagnosing diseases, understanding the pathology of diseases, and as a as a prognostic indicator.

Microarrays are the basis for various high-throughput technologies used in molecular biology and in medicine. Microarrays may include thousands of microscopic spots of DNA oligonucleotides, proteins, antibodies, or other chemical compounds, to be exposed to a sample. In the case of oligonucleotide microarrays, each spot contains a picomole quantity of a specific DNA sequence, such as short sequence derived from a gene or other DNA element for use as a probe for hybridizing to nucleic acids present in a sample. Probe-target hybridization is usually detected and quantified by fluorescence-based detection of fluorophore-labeled targets to determine the presence and relative abundance of nucleic acid sequences in the target.

Protein microarrays, sometimes referred to as protein binding microarrays, are solid substrates upon which different protein molecules are affixed at defined locations. Protein microarrays can be used to identify protein-protein interactions, to identify the substrates of protein kinases, to identify the targets of biologically active small molecules, and the like. The most common protein microarray is the antibody microarray, where antibodies are spotted onto a chip and used to detect proteins and/or antigens that bind to the antibodies. Conventional protein microarrays utilize similar reagents and methods as used in conventional discrete protein binding assays.

Enzyme-linked immunosorbent assays (ELISA) are mainly in immunology to detect the presence and quantify the amount of an antibody or an antigen in a sample. In an ELISA procedure, an unknown amount of antigen is affixed to a surface, and then a specific antibody is washed over the surface so that it can bind to the antigen. This antibody is linked to an enzyme, and in the final step of the assay, a substrate for the enzyme is convert to a detectable signal. In the case of fluorescence ELISA, antigen/antibody complexes fluoresce so that the presence and amount of antigen can be determined.

Many of these and other biological assays utilize a fluorophore to detect the presence of a biological target. Exemplary fluorophores are fluorescein isothiocyanate (FITC), rhodamine, tetramethyl rhodamine, Texas Red, cyanine (Cy2), indocarbocyanine (Cy3), and indodicarbocyanine (Cy5), although many other exist. While reasonably effective for some applications, organic fluorophores have significant disadvantages, including susceptibility to irreversible photobleaching and chemical/biological degradation, which limits their use in long-term time-resolved experiments and certain imaging techniques. In addition, some biological assays use fluorophores that interact only indirectly with a biological target, as in the case of conjugated “secondary” antibodies. Such assays require multiple reagents and binding steps, making the methodology complicated and time consuming.

Accordingly, a need exists for more effective compositions and methods for labeling cells for diagnostic, pathological, forensic, and other analysis.

### SUMMARY

The following aspects and embodiments thereof described and illustrated below are meant to be exemplary and illustrative, not limiting in scope.

In one aspect, a method for labeling a biological target is provided. The method comprises, in one embodiment, providing a biological target for labeling and incubating the biological target with a single-reagent labeling composition. The composition comprises, in one embodiment, (i) a nanoparticle core structure, (ii) a targeting molecule specific for the biological target, and (iii) at least one luminescent component. By incubating the target and the composition, labeling of the biological target with the single-reagent labeling composition is achieved.

In some embodiments, the targeting molecule is attached to the nanoparticle and the at least one luminescent component is attached to the nanoparticle.

In some embodiments, the targeting molecule is attached to the nanoparticle and the at least one luminescent component is attached to the targeting molecule,

In some embodiments, the luminescent component is attached to the nanoparticle and the targeting molecule is attached to the at least one luminescent component.

In some embodiments, the nanoparticle core structure is selected from the group consisting of a dendrimer, a liposome, a metal oxide, a silicon oxide (silica), a lipid micelle, a lentivirus, a plastic bead, and a polymer micelle.

In a particular embodiment, the nanoparticle core structure is a liposome.

In some embodiments, the at least one luminescent component is a fluorescent nanocrystal.

In particular embodiments, the fluorescent nanocrystal is a quantum dot, a quantum rod, or a quantum wire.

In some embodiments, the targeting molecule is an antibody, a fragment of an antibody having binding specificity to the biological target, a nucleic acid, a receptor, a ligand, or more generally, a target on a cell.

In some embodiments, the method further comprising sonicating the biological target following incubation with the labeling composition to remove non-specifically-bound labeling composition.

In another aspect, a composition for labeling a biological target is provided. The composition comprises a single labeling reagent comprising a nanoparticle core structure having a plurality of binding sites for multiplex attachment of at least one targeting molecule, and at least one luminescent component having one or more preselected wavelengths.

In some embodiments, the nanoparticle core structure is selected from the group consisting of a dendrimer, a liposorne, a metal oxide, a silicon oxide (silica), a lipid micelle, a lentivirus, a plastic bead, and a polymer micelle.

In a particular embodiments, the nanoparticle core structure is a liposome,

In some embodiments, the at least one luminescent component is a fluorescent nanocrystal.

In some embodiments, the fluorescent nanocrystal is a quantum dot.

In some embodiments, at least one targeting molecule is an antibody (polyclonal antibody or monoclonal antibody). One preferred antibody is one having specificity for a HER2 receptor.

In some embodiments, the targeting molecule is a fragment of an antibody having binding specificity to the biological target.

In some embodiments, the targeting molecule is a nucleic acid, a receptor, or a ligand.

In some embodiments, the fluorescent nanocrystal is modified with carboxyl groups to facilitate attachment to the nanoparticle core structure.

In another aspect, a composition for labeling a biological target is provided. The composition comprises a single labeling regent comprising a nanoparticle core structure, at least one targeting molecule, and at least one luminescent component having one or more preselected wavelengths.

In some embodiments, the targeting molecule is attached to the nanoparticle and the at least one luminescent component is attached to the nanoparticle.

In some embodiments, the targeting molecule is attached to the nanoparticle and the at least one luminescent component is attached to the targeting molecule.

In some embodiments, the at least one luminescent component is attached to the nanoparticle and the targeting molecule is attached to the at least one luminescent component.

In another aspect, a method for reducing non-specific labeling of a biological sample is provided. The method comprises using a composition as described herein and sonicating the biological material following binding of the nanoparticle labeling reagent to remove non-specifically bound nanoparticle labeling reagent.

In yet a further aspect a method for detecting a biological target is provided, comprising contacting the biological target with a composition as described, herein.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1B illustrate a prior art, conventional method for performing an immunoassay.

FIGS. 2A-2F illustrate exemplary polymer or dendrimer-based nanoparticle labeling compositions.

FIGS. 3A-3B illustrate exemplary liposome or metal particle-based nanoparticle labeling compositions.

FIG. 4 illustrates an embodiment of the present method for performing an immunoassay.

FIG. 5 illustrates an exemplary labeling method using a nanoparticle labeling reagent.

### DETAILED DESCRIPTION

### I. Definitions

The following definitions are provided for clarity. Words and terms not defined should be accorded their ordinary meaning as use in the art. Note that the single articles “a,” “an,” and “the” encompass the plural, unless otherwise specified.

As used herein, a “nanoparticle” is a structure having at least one dimension between about 1-1,000 nm in one or more dimensions. Exemplary nanoparticles include but are not limited to dendrimers, liposomes, semiconductor crystals (e.g., quantum dots), metal particles, magnetic particles, carbon tubes, Bucky balls, quantum rods (QRs), quantum wires (QWs), and other nanoparticles.

As used herein, a “dendrimer” refers to a branched polymer structure, preferably a synthetic polymer structure.

As used herein, a “liposorne” is a self-enclosed vesicle comprised of vesicle-forming lipids, such as a phospholipid.

As used herein, “multiplex attachment” refers to the ability to bring together into a stable structure a preselected number and type of specified components in a modular, tunable, customizable manner to produce a variety of different conjugate structures using a selection of substitutable or interchangeable components.

As used herein, a “biological target” refers to a molecule known or suspected of being present in a biological sample and which can be detected using, e.g., an antibody, a receptor or ligand, a nucleic acid, or other targeting molecule that attaches with specificity to the biological target.

As used herein, “physically or chemically attached” means attached through covalent, ionic, hydrostatic, or other chemical bonds, including but not limited to sulfide and amide bonds.

### II. Nanoparticle Labeling Composition

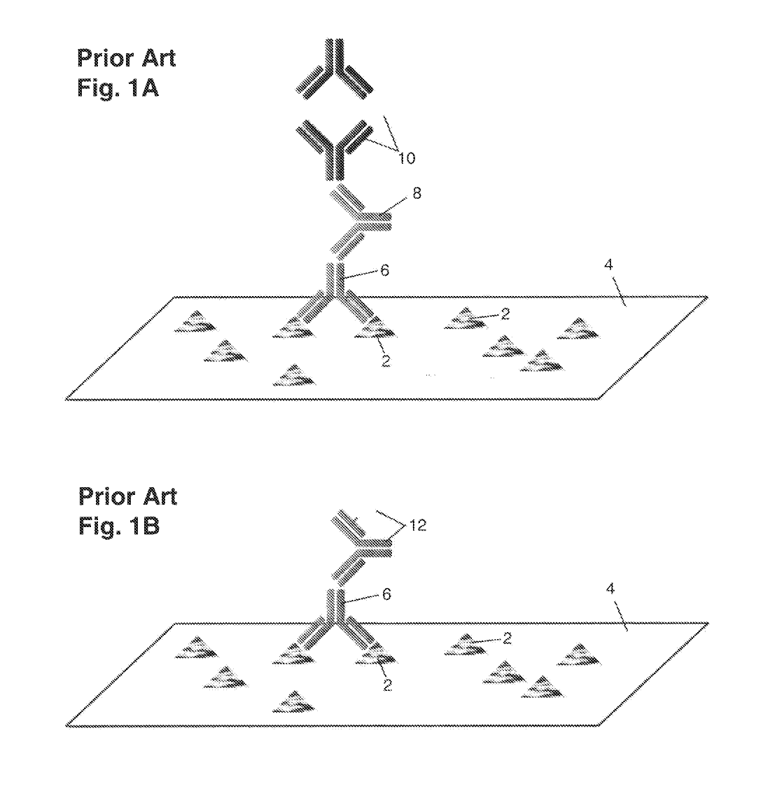

In one aspect, a composition for labeling a biological target is provided. The composition, in one embodiment is comprised of a nanoparticle labeling reagent conjugate that includes a nanoparticle core structure, with one or more luminescent components and one or more labeling agents, which may be physically or chemically attached to form a conjugate. The composition allows specific and efficient labeling of a biological target using a single labeling reagent. Additional reagents, such as secondary antibodies, antibody-enzyme conjugates, enzyme substrates, and the like, can be additionally used, but, as will be appreciated, are not required for labeling and detection of the biological target. These and other features are described with reference to FIGS. 1A-1B (prior art) and Comparative Example 1, wherein prior art, conventional reagents and methods for labeling a biological target are illustrated and described. A study of Comparative Example 1, in conjunction with FIGS. 1A-1B, shows the multiplicity of reagents and steps required to perform conventional labeling methods, and illustrate the need for a simpler, more efficient approach.

The present compositions and methods label a biological target with high efficiency, while avoiding over-staining, and allow multiplexing of luminescent components and labeling molecules. The present labeling compositions also resist photo-bleaching, and provide stable emissions for demanding analyses, long term storage and archiving, In some embodiments, sonication of a labeled biological target is used to reduce non-specific binding, further decreasing background and improving the labeling quality.

These and other features of the compositions and methods are described in detail below, where Section A, describes exemplary labeling reagent compositions, and Section B, exemplary methods of use.

A. Nanoparticle Labeling Reagent Compositions

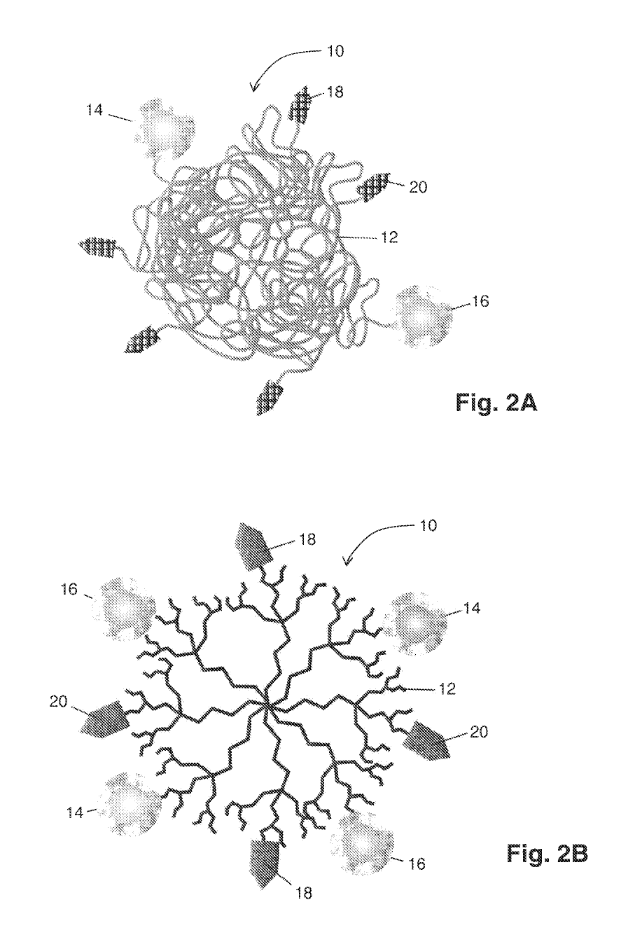

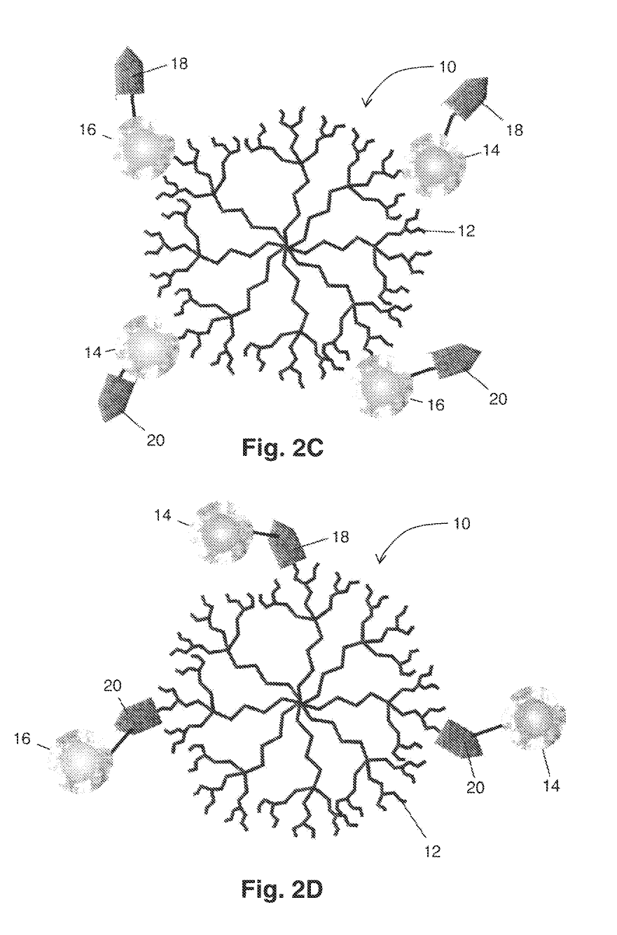

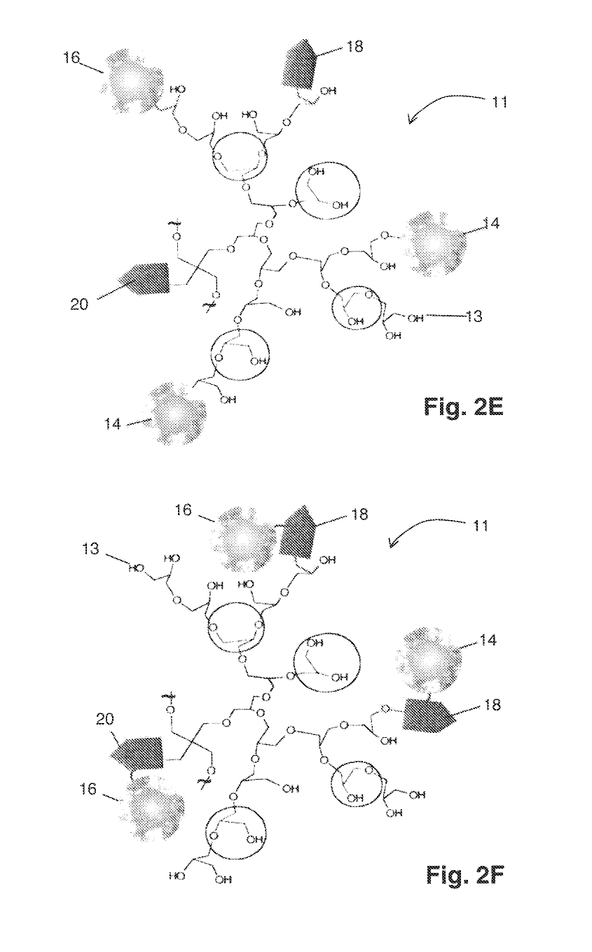

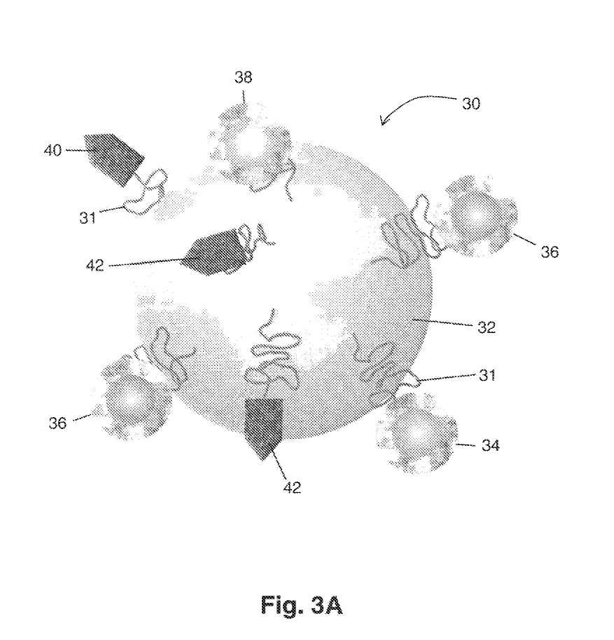

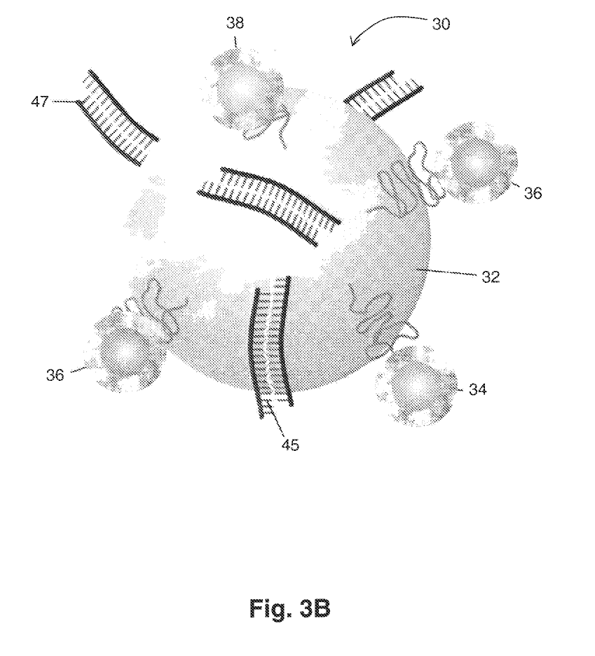

Embodiments of the present nanoparticle labeling reagent 10 are illustrated in FIG. 2A-2F and FIGS. 3A-3B. In first embodiments, the labeling reagent is comprised of a dendrimeric nanoparticle core structure 12 and one or more (i.e., at least one) luminescent component, such as representative luminescent components 14, 16; and one or more (i.e., at least one) targeting molecule, such as representative targeting molecules 18, 20 (FIGS. 2A-2D). In some embodiments, luminescent components 14, 16 are attached to nanoparticle 12, and targeting agents 18, 20 are attached to nanoparticle 12, via the same or different type of chemical or physical interactions (FIGS. 2A-2B). Such interactions include but are not limited to electrostatic interactions, hydrogen bonding, and covalent bonding. The luminescent components 14, 16 need not be attached to the targeting molecules 18, 20 but can be if desired.

In other embodiments, luminescent components 14, 16 are attached to nanoparticle 12, and targeting agents 18, 20 are attached to luminescent components 14, 16, via the same or different type of chemical or physical interactions (FIG. 2C). In yet other embodiments, targeting agents 18, 20 are attached to nanoparticle 12, and luminescent components 14, 16, are attached to targeting agents 18, 20 via the same or different type of chemical or physical interactions (FIG. 2D).

The nanoparticle core structures 12 in the embodiments illustrated in FIGS. 2A-2D are dendrimeric nanoparticles, comprised of a synthetic or biological polymeric structure, preferably with branching to permit attachment of a plurality of luminescent components and/or targeting agents, for polyvalent presentation of each to a target structure. In one embodiment, a composition in accord with the present subject ma comprised of a plurality of such nanoparticle labeling reagents in solid, semi-solid or liquid form.

In a related embodiment of the nanoparticle labeling reagent 11, luminescent components 14, 16 are attached to hyperbranched polymer nanoparticle 12, and targeting agents 18, 20 are attached to luminescent components 14, 16, via the same or different type of chemical or physical interactions (FIG. 2E). In yet other embodiments, targeting agents 18, 20 are attached to hyperbranched polymer nanoparticle 13, and luminescent components 14, 16, are attached to targeting agents 18, 20 via the same or different type of chemical or physical interactions (FIG. 2F).

Another embodiment of the nanoparticle labeling reagent is illustrated in FIG. 3A, in which the nanoparticle labeling reagent 30 is in the form of a liposomal, silicon oxide, metal, or metal oxide particle 32. Attached to nanoparticle 32 is a plurality of the same or different luminescent components, such as luminescent components 34, 36, and 38, which are representative. Also attached to nanoparticle 32 is a plurality of the same or different targeting molecules, such as representative targeting molecules 40, 42. The luminescent components and/or targeting molecules may be attached via linkers 31 The linkers used to attach different components need not be the same. As above, the luminescent components 34, 36, and 38 need not be attached to the targeting molecules 40, 42 but can be if desired.

Yet another embodiment of the nanoparticle labeling reagent is illustrated in FIG. 3B. As in FIG. 3A, the nanoparticle labeling reagent 30 is in the form of a liposomal, silica, or metal particle 32. Attached to nanoparticle 32 is a plurality of the same or different luminescent components, such as luminescent components 34, 36, and 38, which are representative and a plurality of the same or different targeting molecules, such as representative targeting molecules 45, 47, In this embodiment, the targeting molecules 45, 47 are nucleic acids, such as single stranded or double-stranded nucleic acids. As before, the luminescent components and/or targeting molecules may be attached via the same or different linkers 31, The luminescent components 34, 36, and 38 need not be attached to the targeting molecules 45, 47 but can be if desired, In related embodiments, nucleic acid targeting molecules are used in combination with polymeric/dendrimeric nanoparticles, as in FIGS. 2A-2B.

In related embodiments, luminescent components are attached to a liposomal, silica, or metal nanoparticle and targeting agents are attached to the luminescent components via the same or different type of chemical or physical interactions, or targeting agents are attached to a liposomal, silica, or metal nanoparticle, and luminescent components are attached to the targeting agents via the same or different type of chemical or physical interactions (not shown).

Having described the basic features of the present labeling agents, exemplary nanoparticles, luminescent components, and targeting molecules for use in the present labeling reagents are set forth, below.

1. Luminescent Components

As noted above, the nanoparticle labeling reagent composition includes a luminescent component, preferably a photoluminescent component, An exemplary and preferred photoluminescent component is a nanocrystal of semiconducting materials, such as quantum dots (QDs), quantum rods (QRs), and quantum wires (QWs). QDs, QRs, and QWs have several advantages over conventional fluorescent dyes, including a long luminescent lifetime and near quantitative light emission at a variety of preselected wavelengths. QDs typically contain a semiconductor core of a metal sulfide or a metal selenide, such as zinc sulfide (ZnS), lead sulfide (PbS), or, most often, cadmium selenide (CdSe). The semiconductor core may be capped with tiopronin or other groups or otherwise varied to modify the properties of the quantum dots, most notably to vary biocompatibility. and enhance chemical versatility. The emission wavelengths of nanoparticles may be between about 400 nm and about 900 nm, including but not limited to the visible range, and the excitation wavelength between about 250 nm and 750 nm.

QDs typically have diameters of 1 to about 15 nm, depending on the emission wavelength desired and the particular application for the nanoparticle labeling reagent. In freeze-fracture electron microscopy characterization, the shadow cast by QDs is evidence of their hard-core structure. One or more QDs can be conjugated to a single nanoparticle core structure, which is to be described. The number of QDs attached to a core structure may be at least two, at least three, at least four, or 10 or more, 100 or more, or even 1,000 or more, limited in part by the surface area of the nanoparticle core particle and steric effects of adjacent QDs. The QDs on a particular nanoparticle core structure may be of a single color (i.e., single predominant emission wavelength), or of a plurality of colors.

A selected set of QDs may be attached to a nanoparticle core structure in a multiplexed manner to produce nanoparticle labeling reagents with a “bar code,” i.e., an emission spectra characterized by particular emission wavelengths and intensities (both relative and absolute). Such labeling reagents can be used for, e.g., (i) multi-color (ii) multi-color coding, (iii) multiple parameter diagnosis, and the like.

2. Nanoparticles

The nanoparticle core structure in the nanoparticle labeling reagent serves as the core structure or scaffold, for the luminescent components and labeling agents (described, below) and imparts unique properties to the nanoparticle labeling reagent through its size and chemical composition.

An exemplary nanoparticle is a dendrimer cores structure. Dendrimeric polymers herein, “dendrimers”) are repeatedly branched molecules that include dendrons, dendronized polymers, hyperbranched polymers, and brush-polymers. The core structure of the dendrimer largely determines the overall shape, density, and surface topology, while the surface exposed end functional groups affect solubility, hydrophobicity/hydrophilicity, and generally how the dendrimer interacts with other molecules.

Dendrimers are well-known in the art and described in numerous references, including but not limited to U.S. Pat. Nos. 4,694,064, 4,568,737, 4,507,466, 6,471,968, 4,410,688, and 4,289,872, Buhleier, E. et al. (1978) “Cascade”- and “Nonskid-Chain-like” Syntheses of Molecular Cavity Topologies, Synthesis 155-58; Tomalia, D. et al, (1985) A New Class of Polymers: Starburst-Dendritic Macromolecules (No. 1), pp,117-32; George, R. et al. (1985) J. Org. Chem. 50, 2003-04; Hawker, C. and Fréchet, J. (1990) J. Am. Chem. Soc. 112:7638; Hecht, S. et al. (2001) Angew. Chem, Int. Ed. 40:74; Fréchet, J. et al. (2001) Dendrimers and Other Dendritic Polymers, John Wiley & Sons, Ltd. N.Y., N.Y.; Fischer, M. and Vogtle, F. (1999) Angew. Chem. Int. Ed. 38:884; Tomalia et al. (1990) Chem. Int. Ed. Engl. 29:5305; and Yin et al. (1998) J. Am. Chem. Soc., 120:2678, which are hereby incorporated by reference in their entirety.

Particular dendrimers are the dense polyamidoamine (PAMAM) star polymers described in U.S. Pat. Nos. 4,507,466, 4,558,120, 4,568,737, 4,587,329, and 5,338,532; poly(etherhydroxylamine) (PEHAM) dendrimers (WO 2008/030591); the dense star dendrimers with a hydrophobic outer shells described in U.S. Pat. Nos. 5,387,617, 5,393,797, and 5,393,795; the rod-shaped dendrimers described in U.S. Pat. No. 4,694,064; the hydrolytically stable dendrimers described in U.S. Pat. No. 4,631,337; the non-crosslinked, polybranched polymers with a comb-burst configuration described in U.S. Pat. No, 5,773,527; the polybranched, high-molecular dendimers described in U.S. Pat. No. 5,631,329; and the amino terminated, antibody-conjugated dendrimers described U.S. Pat. No. 5,527,524. Dendrimers are generally non-toxic when administered intravenously (e.g., Roberts et al. (1996) J. Biomed. Mat. Res. 30:53 and Bourne et al. (1996) J. Magn. Reson. Imag. 6:305), although the particular size, shape, and end group composition of a dendrimer is likely to affect toxicity.

Dendrimer structure and other physical properties may be characterized by a number of techniques including Put not limited to electrospray-ionization mass spectroscopy, high performance liquid chromatography (HPLC), size exclusion chromatography, laser light scattering, capillary electrophoresis, gel electrophoresis, and ¹³C nuclear magnetic resonance spectroscopy. Such characterization may be to ensure uniformity in a dendrimer preparation to or select for a subpopulation of dendrimers having preselected properties, and/or for chemical/physical characterization.

The nanoparticle core structure in the nanoparticle labeling reagent may also be a hyperbranched polymer. Hyperbranched polymers are similar to dendrimers. However, whereas a dendrimer is a “perfect” molecule, in which substantially the entire molecule is branched, a hyperbranched polymer is an “imperfect” molecule, in that it may include linear sections. In addition, a dendrimer typically includes a multi-functional core, repeated branching units, and surface functional groups, and is typically synthesized in a multi-step process, while a hyperbranched polymer is a less complex structure synthesized in a single step reaction from functional monomers, or polycondensation, ring-opening multibranched polymerization, self-condensing vinyl polymerization, etc. Exemplary hyperbranched polymers include but are not limited to hyperbranched polyglycerols, polyamidoamines, polyamines, polyethers, polyesters, polyphenylenes, polyamides, polycarbonates, poly(ether ketone)s, polyurethanes, polycarbosilanes, poly(acetophenone)s, and polysiloxanes, etc.

Another exemplary nanoparticle core structure is a liposome. Liposomes are self-enclosed vesicles formed from amphipathic lipids, such as phospholipids. Typical phospholipids used in formation of liposomes are phosphatidylethanolamine (PE) and phosphatidylserine (PS), phosphatidylcholine (PC), phosphatidylinositol (PI), phosphatidic acid. Other lipids commonly used in liposomal particles include sphingomyelin, glycolipids, cerebrosides, and sterols, such as cholesterol. Liposomes may optionally or additionally include cationic lipids such as 1,2-dioleyloxy-3-(trimethylamino) propane (DOTAP), N-[1-(2,3,-ditetradecyloxy)propyl]-N,N-dimethyl-N-hydroxyethylammonium bromide (DMRIE), N-[1-(2,3,-dioleyloxy)propyl]-N,N-dimethyl-N-hydroxy ethylammonium bromide (DORIE). N-[1-(2,3-dioleyloxy) propyl]-N,N,N-trimethylammonium chloride (DOTMA), 3 [N-(N′,N′-dimethylaminoethane)carbamoly]cholesterol (DC-Chol), or dimethyldioctadecylammonium (DDAB).

The liposomes can optionally have an external surface coating of hydrophilic polymers, generally included in the liposomes by incorporation of a vesicle-forming lipid having a covalently attached hydrophilic polymer. The presence of the hydrophilic polymer provides functional groups on the surface of the liposome for interaction with solvents and other molecules, and for points of attachment of luminescent components and/or targeting molecules. The hydrophilic polymer coatina also provides shielding effect for nanoparticles to reduce non-specific interactions with biological and immune systems. Numerous lipids can be derivatized using hydrophilic polymers, including but not limited to distearoyl phosphatidylethanolamine (DSPE), Hydrophilic polymers suitable for use include polyvinylpyrrolidone, polyvinylmethylether, polymethyloxazoline, polyethyloxazoline, polyhydroxypropyloxazoline, polyhydroxypropylmethacrylamide, polyrnethacrylamide, polydimethylacrylamide, polyhydroxypropylmethacrylate, polyhydroxyethylacrylate, hydroxymethylcellulose, hydroxyethylcellulose, polyethyleneglycol, polyaspartamide, and hydrophilic peptide sequences. Such polymers may be in the form of homopolymers, block polymers, or random copolymers. Preferred hydrophilic polymers suitable for use in derivatizing vesicle-forming lipids are polyethyleneglycol (PEG) and its methoxy, ethoxy or ethoxy-capped analogues, preferably having molecular weights between 500-10,000 daltons and 120-20,000 daltons, respectively. The percent of hydrophilic polymer present in a liposome composition may vary from about 1 to about 20 mole-percent. Preparation of vesicle-forming lipids derivatized with hydrophilic polymers has been described, for example in U.S. Pat. No. 5,395,619.

Lipids may also include residual amount of solvents, such as amphipathic solvents including polyethyleneglycol, ethanol, and other aliphatic solvents, or surfactants, such as sodium dodecyl sulfate (SDS): sodium dodecyl ether sulfate (SDES), Triton X-100, and dodecyl betaine (D-Bet).

Liposomes are often characterized based on their phase transition temperatures (i.e., from solid to liquid form) which depend on the components vesicle-forming lipids in the liposome. For the purpose of the present compositions and methods, preferred liposomes have a phase transition temperatures between about 5-70° C.

Other exemplary nanoparticle structures for use in the present nanoparticle labeling reagent include, but are not limited to, carbon nanotubes. Bucky balls, metal and metal oxides particles (including magnetic particles), silicon oxides (silica) particles, polymer micelles, plastic nanobeads, and virus particles and capsids. Exemplary viruses are retroviruses (including lentiviruses), picornaviruses, flaviviruses, pox viruses, herpes viruses, potiviruses, and other plant and animal viruses.

The term “nanoparticle” technically encompasses QDs. However, since QDs have a specific function as the luminescent component of the present nanoparticle labeling reagents, preferred nanoparticle core structures are not QDs.

3. Targeting Molecules

The nanoparticle labeling reagent includes one or more targeting molecules that binds specifically to a biological target. Biological targets may be proteins (including glycoproteins), nucleic acids, carbohydrates, lipids (including glycolipids), or combinations, thereof. Biological targets may be at least partially embedded in a membrane, secreted, or soluble in the cytoplasm. Where the biological target is present on the surface of a cell, it is generally accessible to targeting molecules without fixing the cells, as in the case of cells in suspension, including cells in vivo. Where the cellular target is present inside the cell, it is generally accessible after fixing the cell, optionally in combination with lysing the cell to release its contents, or solublizing the membranes to expose its contents.

Where the biological target is a protein, the targeting molecules may be an antibody, an analog of the natural binding partner of the protein, or a substrate for the protein. The affinity of targeting molecules for the cellular target is not critical but should be greater than about 10⁻⁶ molar (M), greater than about 10⁻⁷ M, greater than about 10⁻⁸ M, greater than about 10⁻⁹ M, greater than about 10⁻¹⁰ M, or even greater than about 10⁻¹¹ M.

Antibodies include polyclonal antibodies, monoclonal antibodies, synthetic antibodies, antibodies, or immunogenically active fragments, or derivatives, thereof. Exemplary fragments are F(ab′)₂, Fab′, scFv, and the like, Derivative include pegylated and other modified antibodies. Antibodies and fragments may be chimeric, humanized, humaneered, single-chain, or other wise modified to modulate their affinity and/or avidity for a cellular target, immunogenicity in an organism, half life, or other physical properties. Exemplary anitibodies are drugs such as trastuzumab, cetuximab, bevacizumab, rituximab, ranibizumab, and fragments and derivatives, thereof.

Where the biological target is a nucleic acid, the targeting molecules may be nucleic acid probes, including DNA, RNA, and nucleic acids including synthetic bases, thiodiester bonds, end-capping groups, and other modifications. Ideal probes are from about 15 to about 100 nucleotides in length, although longer nucleotides may produce acceptable results. Exemplary nucleotides probes are 15, 20, 25, 30, 35, 40, 45, 50, 55, 60, 65, 70, 75, 80, 85, 90, 95, and 100 in length.

Targeting molecules also include receptors, ligands, peptide or small-molecule binding partners, substrates, and/or inhibitors for preselected receptors, proteases, kinases, phosphatases, polymerases, growth factors, cell cycle proteins, enzymes involved in energy metabolism, structural proteins, proteins involved in mitosis or cytokinesis, and the like. An exemplary small-molecule targeting compound is folate, which targets the folate receptor.

Most any biological target that can be detected using a conventional biological assay can be detected using the present nanoparticle labeling reagents, albeit with superior sensitivity, increased stability, and reduced non-specific binding, Exemplary biological targets that can be detected using antibodies, nucleic acids, or other targeting molecules, include but are not limited to HER2. retinoblastoma gene product (Rb), cyclin A. nucleoside diphosphate kinase/nm23, telomerase, Ki-67, cyclin D1, proliferating cell nuclear antigen (PCNA), p120 (proliferation-associated nucleolar antigen), thyroid transcription factor 1 (TTF-1), VEGF, surfactant apoprotein A (SP-A), nucleoside nm23, melanoma antigen-1 (MAGE-1), mucin 1, surfactant apoprotein B (SP-B), ER related protein p29 and melanoma antigen-3 (MAGE-3). thrombomodulin, CD44v6, E-Cadherin, human epithelial related antigen (HERA), fibroblast growth factor (FGF), heptocyte growth factor receptor (c-MET), BCL-2, N-Cadherin, epidermal growth factor receptor (e.g., EGFR, ErbB2, ErbB3, ErbB4), glucose transporter-3 (GLUT-3), BCL-2, p120 (proliferating-associated nucleolar antigen), Fos, Jun, Myc, Ras, vascular epidermal growth factor receptor (VEGFR), folate, human insulin receptor, insulin-like growth factor I receptor (IGF-IR), transferrin, CD44, CD19, CD20, GD2, α-v β 3-integrin, β1 integrin, anti-ED-B B-fibronectin scFv, vasopressin, bradykinin, aminopeptidase N, vasoactive intestinal peptide receptor, multiple drug resistance pumps (MDRs), and various P-glycoproteins (PGPs), lipoproteins, and glycolipids.

The nanoparticle labeling reagents can be multiplexed by use of a defined set of labeling molecules on each nanoparticle core structure to produce labeling reagents with complex binding specificities, e.g., involving more than one biological target. A single nanoparticle may have attached different antibodies, nucleic acids, ligands, small molecules, or the like, or combinations, thereof, to produce labeling reagents with specificity to more than one biological target and/or to more than one type of biological macromolecule, For example, a single scaffold or nanoparticle core structure can have attached as labeling agents a first antibody specific for a first antigen and a second antibody specific for a second antigen to identify a two or more biological targets simultaneously, to preferentially identify biological material having both targets in sufficient proximity to contact a single nanoparticle labeling reagent, or variations, thereof. Similarly, a nanoparticle labeling reagent may be multiplexed with, e.g., an antibody specific for a protein expressed in a disease state and a nucleic acid specific for a gene mutation associated with the disease state.

In this manner, numerous combinations of labeling molecules can be attached to a single nanoparticle core structure to produce nanoparticle labeling reagents with complex binding specificities.

4. Formation of Nanoparticle Labeling Reagent

The nanoparticle labeling reagent can be prepared using any number of techniques, and no particular chemical method is required. Methods for attaching “components” such as QDs, antibodies, and other molecules to dendrimers, liposomes, metal particles, and other nanoparticles are known in the art, Such methods may involve derivitization of the nanoparticles, and/or components, with functional groups such as carboxyl, alcohol, amine, amino, thiol, disulfide, urea, or thiourea groups, which then allow chemical linkage of t nanoparticles and components using conventional methods. Following derivitization, assembly of these components often proceeds readily, and may be referred to as “self assembly.”

Methods for derivatizing and attaching QDs, dendrimers, liposomes, metal particles, and other particles can be found, e.g., in Rhyner. M. N et al. (2006) Nanomedicine 1:209-17; Jamieson, T. (2007) Biomaterials 28:4717-32; Iga, A. M. (2007) J. Biomed. Biotech. 2007:76087-97; Zhou, M. et al. (2007) Bioconjugate Chemistry 18:323-32; Tortiglione, C. et al. (2007) Bioconjugate Chemistry 18:829-35; Selvan, S. T. et al. (2007) Angewandte Chemie International Edition 46:2448-52; Kampani, K. et al. (2007) J. Virological Methods 141:125-32; Medintz, I. L. et al. (2007) Nano Letters 7;1741-48; de Ferias, P. M. A. et al. (2005) J. Microscopy 219:103-08; Gao, X. et al. J. Biomedical Optics. 7:532-37; Tan, W. B. et al. (2007) Biomaterials 28:1565-71; Allen, T. M. et al. (1995) Biochim. Biophys. Acta. 1237:99-108; and Hansen, C. B. et al. (1995) Biochim. Biophys. Acta. 1239:133-44; in the references cited above and herein; and in U.S. Pat. Nos. 7,138,121, 7,133,725, 7,112,337, 7,103,863, 6,369,206, 5,861,319, 5,714,166, and 5,468,606.

A particular method for forming nanoparticle labeling reagents uses 1-ethyl-3-(3-dimethylaminopropyl)carbodiimide, as described by Sheehan, J. and Hlavka, J. ((1957) J. Am. Chem. Soc. 79: 4528-429).

B. Methods of Using a Composition of Nanoparticle Labeling Reagents

In other aspects, a method for labeling a biological target using a nanoparticle labeling reagent composition is provided. In particular embodiments, the method is for detecting cells or a cellular structure comprising a biological target. The method is applicable to a number of different labeling protocols that rely on interaction between a preselected biological target and a targeting molecule, including but not limited to, immunohistochemistry (IHC) assays, fluorescence in situ hybridization (FISH) assys, fluorescence-activated cell sorting (FACS, or flow cytometry) assays, microarray assays, enzyme-linked immunosorbent assays (ELISA), immunoprecipitation assays, immunoblot assays (i.e., western blots), and the like. These and other biological assays are described in, e.g., Sambrook et al., (1989) Molecular Cloning: A Laboratory Manual, Cold Spring Harbor Labs Press.

In some cases, the targeting molecule is preferably an antibody specific for a biological target, In other cases, the targeting molecule is a nucleic acid that is at least partially complementary to a target nucleic acid. In other cases, the targeting molecule is a receptor, ligand, or other binding partner specific for a biological target. More than one of each type of targeting molecule (e.g., antibody, nucleic acid, receptor, ligand, etc.), and/or more than one type of targeting molecule, can be present on a single nanoparticle, thereby producing labeling composition with complex binding specificy.

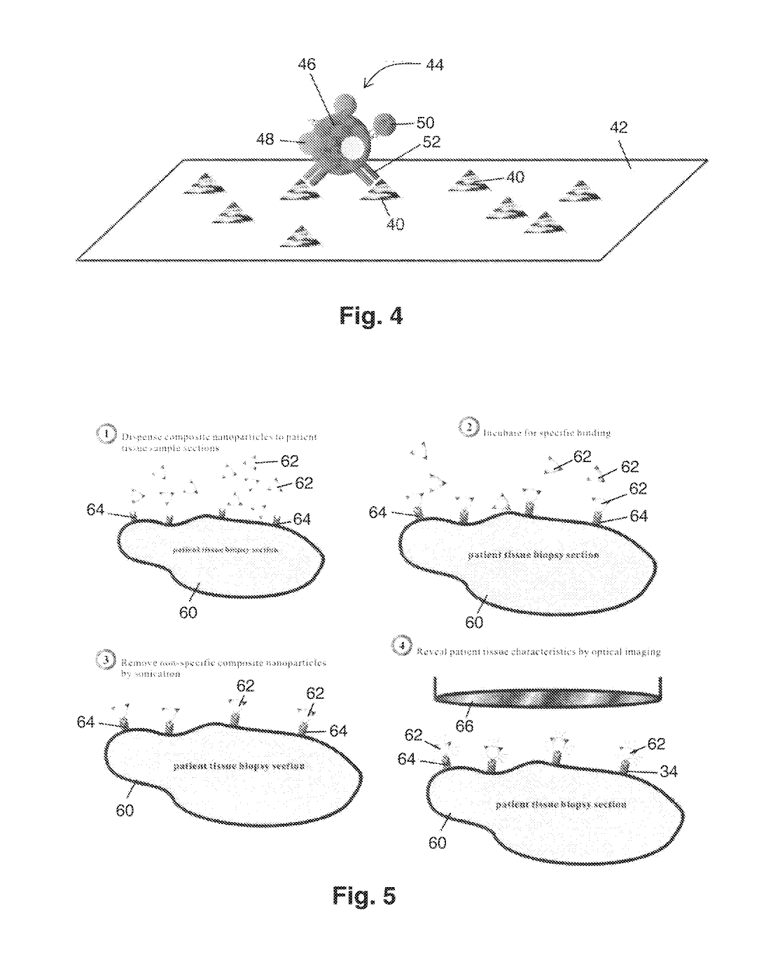

A method for labeling a biological target is generally illustrated in FIG. 4 and supported with experimental details in Example 1. This exemplified assay is an IHC assay, although the principles apply to other assays. With reference to FIG. 4, tissues, cells, cell material, or other material containing or suspected of containing a biological target 40 are provided for labeling, The material containing a biological target 40 may be immobilized on a solid support 42, such as a glass slide, well of a multi-well plate, or the like, The material containing a biological target 40 is incubated in the presence of a nanoparticle labeling reagent 44, comprising a nanoparticle support structure 46, at least one luminescent component, such as fluorescent nanocrystals 48, 50, which may be the same or different luminescent components, and a labeling molecule 52 with specific binding affinity for a biological target in the material 40. Together the components 46, 48, 50, and 52 are attached to form a single labeling reagent 44.

Referring now to FIG. 5, tissues, cells, or cell material 60 that contains or is suspected of containing a biological target 64 are provided for labeling as in the form of, e.g., a tissue section, blood smear, biopsy sample, monolayer or suspension of cells, or similar clinical, pathological, or research samples. The cell material 60 may be washed in a buffer such as phosphate-buffered saline (PBS), tris-buffered saline (TBS), phosphate buffer, 4-(2-hydroxyethyl)-1-piperazineethanesulfonic acid (HEPES) buffer and the like. In some embodiments, the cell material 60 may be fixed, e.g., using glutathione, methanol, formaldehyde, anti-fade mounting medium, and the like, while in other embodiments the cells are not fixed. The cell material 60 may be on a solid support, such as a flask, dish, or slide, in suspension, in a fluorescent assay cell sorting device, in vitro, in vivo, or ex vivo.

The cell material 60 is incubated in the presence of a nanoparticle labeling agent 62 as described herein. In this and other embodiments of the method, the large size of the labeling agent 62 precludes overstaining caused by non-specific binding of the labeling agent to the cell surface. The labeling agent 62 can be multiplexed to allow the attachment of multiple luminescent components to one or more antigens 64, there producing an intense signal from each specific binding event.

Unbound nanoparticle labeling reagents 62 can be washed away from the cell material 60 using a wash buffer as above, Wash buffers may additionally include any number of salts, surfactants, chelating agents, or other components to promote the removal or non-specifically bound labeling agents. Examples of suitable wash buffers are PBS and other phosphate buffers, TBS and other Tris buffers, Hepes, and other buffers and washes used for cell culture, histology, forensics, clinical diagnosis and treatment, and the like. Further reduction of non-specific binding can be achieved using sonication, as described, below.

The remaining, specifically-bound nanoparticle labeling reagents 62 are capable of producing a detectable signal without the addition of further labeling reagent such as an antibody, enzyme, antibody conjugate, substrate, or luminescent reagent, thereby functioning as a “single-reagent” labeling reagent (ignoring incidental reagents such as fixing solutions, wash buffers, and the like). When exposed to light energy at the excitation wavelength, the bound nanoparticle labeling reagents producing a signal detectable by, e.g., an optical imaging device 66.

As noted above, while the nanoparticle labeling reagent and method are exemplified for use in immunohistochemistry assays, they are also suitable for use with numerous other assays that rely on the detection of a biological target with a labeling molecule.

C. Sonication to Reduce Non-specific Binding

Sonication refers to the process of applying sound energy to an object to agitate particles, therein. Sonication can be used to speed dissolution of materials, e.g., by breaking intermolecular bonds, to provide energy to encourage chemical reactions, to degas liquids (degassing), to disrupt cellular membranes, and the like, One common application of sonication is to clean laboratory equipment, jewelry, tools, and other items. The frequency of sound used for sonication is typically in the ultrasound range, i.e., above the maximum frequency detectable by humans, which is about 20,000 Hertz (20 kHz) in young humans.

Sonication may be used to reduce non-specific binding of nanoparticle labeling reagents to reduce non-specific binding and enhance positive-negative contrast. Without being limited to a theory, it is believed that the relatively large size of the present nanoparticle labeling reagents. i.e., compared to conventional antibody reagents, dyes, and stains make, the nanoparticle labeling reagents sensitive to ultrasound energy, which can be used to dissociate non-specifically-bound labeling reagents from cells and assay surfaces. Non-specifically bound labeling agents are more readily dissociated because they are only weakly attached to cells or assay surfaces, while specifically bound labeling agents resist dissociation. Moreover, because the resonance frequency of a particle changes when is becomes attached to another particle, such as a specific target on a cell, bound labeling agents are less affected by ultrasound energy than unbound particles.

Ultrasound frequencies for use in reducing non-specific binding of the present labeling reagents are in the range of about 15 kHz to about 200 kHz, and typically in the range of about 25 kHz to about 40 kHz, although frequencies outside these ranges may provide satisfactory results. A single predominant ultrasound frequency or a plurality of different ultrasound frequencies may be used to remove non-specifically bound labeling reagents. A predominant ultrasound frequency may be accompanied by any number of overtones and harmonics, Ultrasound procedures are generally performed in an acoustically isolated chamber or with suitable protective apparatus. In one embodiment, sonication is performed in a miniaturized incubator customized for the slides or biochips containing tissue sections, cellular samples, or other biological samples.

The particular ultrasound frequency or frequencies may be selected by empirically determining the frequency or frequencies that provide optimum background reduction. Alternatively, the particular ultrasound frequency or frequencies may be selected by estimating the resonance frequency of a particular, unbound labeling reagent, and applying ultrasound energy of an appropriate frequency.

Nonspecific labeling is a common problem with many forms of fluorescence-based immunostaining, including those involving nanoparticles. Sonication reduces nonspecific labeling, thereby reducing background and improving contrast enhancement. Sonication can be used to reduce non-specific binding in various types of biological assays, and is not limited to the present nanoparticle labeling reagents.

An application of the present nanoparticle labeling reagent and methods is detailed in Example 2. in the exemplary nanoparticle labeling reagent, the selected fluorescent components were “red-fluorescing” QDs, the selected nanoparticle was a liposome, and the selected labeling molecules were HER2/ErbB2-specific antibodies. Two slides of cultured cells were prepared, each using a different cell population. The first cells had previously been determined to have a low score (1+) for HER2/ErbB2 and the second had previously been determined to have a high score (3+) for HER2/ErbB2 (data not shown). The first cells were minimally labeled with the labeling reagent, with only a few red-fluorescing QDs being visible on the field. In contrast, most of the second cells were brilliantly labeled with the labeling reagent, demonstrating the low background, specificity, and high signal intensity of the present nanoparticle labeling reagents and methods.

The advantages of sonication were demonstrated in another study, where red-fluorescing QD-liposome-HER2 antibody nanoparticle labeling reagent were subjected to sonication or were not subjected to sonication (Example 3). Sonication effectively removed non-specifically bound nanoparticle labeling reagents. An exemplary sonication device is described in Example 4.

D. Advantages of the Nanoparticle Labeling Composition and Method

The present nanoparticle labeling reagent offer several advantages over conventional dyes and immunolabeling reagents and methods. For example, the modular nature of the nanoparticle labeling reagents supports multiplexing of different luminescent components, different labeling molecules, or both, making possible complex arrays of related labeling reagents for identifying multiple targets simultaneously (e,g., multiplexed immunodetection). The modular platform also supports the addition of other functional molecules and particles.

An advantageous feature of the of the nanoparticle labeling reagents is that they provide “amplification” of target signals as a result of polyvalent binding and multiplied optical signals from clustered nanocrystals. Such multivalency allowing a high degrees of signal amplification, allowing the detection and quantitation of targets present in trace amounts. Many of the advantageous optical features of semiconductor luminescent components, such as QDs, are well known, and include signal stability, near quantitative emissions, and different discrete wavelengths,

Another feature of the present composition and method is that visualization of a nanoparticle labeling reagent can be accomplished following a single binding step, without the need for secondary antibodies, enzymes, conjugates, or reagents for producing color or fluorescence. Therefore, the present “single-reagent, single-binding step” composition and method reduces workup time, allowing higher throughput at less cost.

The intensity and stability of nanoparticle labeling reagents supports high-speed optical scanning of labeled cells and cell material and more quantitative analysis than can be obtained using conventional detection methods. For example, dynamic light scattering (DLS) is a widely used technique for nano/micro-particle sizing and characterization based on the relationship between light scattering and Brownian motion of particles in media (see, e.g., Example 5). The present compositions and methods are fully compatible with DLS and other methods for deconvoluting data obtained using optical scanning.

The nanoparticle labeling reagents are not prone to photobleaching, and such persistent optical properties make them ideal for long term storage of labeled cells or cell material without loss of fidelity over time. The labeling reagents can be made substantially non-toxic, and have a variety of uses for identifying and targeting cells in vitro, in vivo or ex vivo, and in vivo, including diagnosis and treatment of animals.

In some embodiments, the present composition and method further include the feature of using sonication to reduce non-specific binding. The sonication step is particularly useful in reducing background obtained using large labeling reagent (such as the present nanoparticle conjugates) which are most affected by high energy sound waves.

E. Kits of Parts

Nanoparticle labeling reagents may be supplied as part of an assay kit in dry or suspended form, in combination with other kit components for performing assays. Kit components may include, for example, resuspension buffers, wash buffers, fixatives, solvents, counterstains, slides, trays, dishes, swaps, droppers, goggles, and the like. Kits of parts may also include written or electronic instructions for using the reagents. Nanoparticle labeling reagents may also be supplied with an optical imaging device, or other equipment. In preferred embodiments, the optical imaging device can be used for a large number of assays.

While the present labeling reagents and method have been described with reference to several drawings, it will be appreciated that features and variations illustrated or described with respect to different drawings or embodiments can be combined in a single embodiment.

Other applications and implementations will be apparent in view of the disclosure. Such modifications, substitutions and alternatives can be made without departing from the spirit and scope of the invention, which should be determined from the appended claims, All references cited herein are hereby incorporated by reference in their entirety.

The following Examples are provide to illustrate the compositions and methods and are not intended to be limiting.

### III. Examples

The following Examples are provided to illustrate the compositions and methods.

### Comparative Example 1

### Prior Art or Conventional Immunostaining Protocol

Conventional methods of immunostaining are illustrated in FIG. 1A and FIG. 1B. In the method illustrated in FIG. 1A, tissues, cells, or cell material including an antigen 2 of interest are immobilized on a solid support 4, such as a glass slide, well of a multi-well plate, or the like and incubated in the presence of a primary antibody 6 specific for the antigen 2 of interest. After removing unbound primary antibody 6 by washing, secondary antibody 8 is allowed to bind to the primary antibody 6, followed by addition of antibody-color development reagent complexes 10 capable of acting on a substrate to produce a detectable signal that corresponds, indirectly, to the present of the antigen 2 of interest.

In the method illustrated in FIG. 18, tissues, cells, or cell material including an antigen 2 of interest are similarly immobilized on a solid support 4 and incubated in the presence of a primary antibody 6 specific for the antigen 2 of interest. After removing unbound primary antibody 6 by washing, a secondary antibody-color development reagent complex 12 is allowed to bind to the primary antibody 6. The complex 12 is capable of acting on a substrate to produce a detectable signal that corresponds, indirectly, to the present of the antigen 2 of interest.

A detailed protocol for a conventional immunohistochemistry procedure is described, below. This example concerns staining slides coated with HER2 human breast carcinoma cells to visualize the erbB2 receptor.

Part 1:
1. Bake slides in oven at 60° C. for 30 minutes prior to staining
2. Deparaffinize and rehydrate the tissues on the slides:
Xylene wash for 5 minutes three times
100% EtOH wash for 5 minutes twice
95% EtOH wash for 2 minutes twice
70% EtOH wash for 2 minutes twice
dH₂O wash for 5 minutes once
PBS wash for 5 minutes twice
3. Incubate with Ficin (proteolytic enzyme) for 10 minutes at 37° C.
4. Wash in PBS for 3.5 minutes three times
5. Block in 3% H₂O₂ for 15 minutes
6. Wash in PBS for 3.5 minutes three times
7. Incubate with normal horse serum for 30 minutes at room temperature
8. Incubate with erbB2 antibody (primary antibody) overnight at 4° C.

Part 2:
9. Wash off coverslips with PBS for 8 minutes
10. Wash in PBS for 3.5 minutes twice
11. Incubate with biotinylated horse anti-mouse (secondary antibody)or 30 minutes at room temperature
12. Wash in PBS for 3.5 minutes three times

Part 3:
13. Incubate with ABC reagent for 30 minutes at room temperature
14. Wash in PBS for 3.5 minutes three times
15. Incubate with DAB/H₂O₂ (color development reagents) for 5 minutes at room temperature
16. Wash in running tap water for 3 minutes

Part 4:
17. Counterstain with Hematoxylin for 1 minute
18. Wash in running tap water for 3 minutes
19. Differentiate in 1% Acid Alcohol for 3 quick dips
20. Wash in running tap water for 3 minutes
21. Blue in Scott's water for 1 minute
22. Wash in running tap water for 3 minutes
23. Dehydrate through graded alcohol and clear in xylene
24. Coverslip with Permount
25. erbB2 level of expression scored by pathologists

As can be appreciated, the conventional method is time-consuming, with typical processing times being about two days. Several discrete binding steps are required, e.g., for binding of primary antibody, secondary antibody, colorimetric development agents, and counterstaining. Moreover, the results are generally not quantitative.

### Example 1

### Method and Protocol For Labeling Using Reagent and Method

An exemplary protocol using the present composition and method for labeling cells is described. Slides coated with HER2 human breast carcinoma cells were stained to visualize the erbB2 receptor as follows.

1. Bake slides in oven at 60° C. for 30 minutes prior to staining

2. Deparaffinize and Rehydrate the tissues on slides (see, above)

3. Incubate with Ficin for 10 minutes at 37° C.

4. Wash in PBS for 3.5 minutes three times

5. Block in 3% H₂O₂ for 15 minutes

6. Wash in PBS for 3.5 minutes three times

7. Incubate with normal horse serum for 30 minutes at room temperature

8. Incubate with the present nanoparticle labeling reagent for 2 hours at 4° C.

9. Sonicate in PBS for 5 minutes

10. Apply coverslip with Permount

11. Inspect and readout by automated high-speed scanning instrument

Relative to Comparative Example 1, this protocol eliminates Steps 8 through 24 of the conventional protocol. Required processing time is approximately 4 hours and may be performed by a medical/biological laboratory assistant with general bench experience. A single reagent replaces the primary antibody, secondary antibody, colorimetric/fluorescent labeling agent, and even counterstaining.

### Example 2

### Exemplary Nanoparticle Labeling Reagent and its Use

An exemplary red-fluorescing QD-liposome-HER2 antibody nanoparticle labeling reagent was produced in a “single-pot” reaction using QDs with carboxyl groups on the outer surface with preformed HER2 irnmunoliposomes in the presence of 1-ethyl-3-(3-dimethylaminopropyl)carbodiimide.

The ratios of QDs, lipids, cholesterol, poly(ethylene glycol), and anti-HER2 antibody/antibody fragment was varied by one or more of the following methods: (i) varying the ratio of the components in the starting mixtures for liposome preparation; (ii) changing the concentration of 1-ethyl-3-(3-dimethylamirtopropyl)carbodiimide and other reaction conditions such as reaction time, pH, and temperature of the reaction environment; and (iii) changing the amount of functionalized lipids and/or antibody-lipid conjugates. Typical ratio of these components were between 1 to 10 QDs per liposomes; 10 to 500 antibodies per liposomes, and 0.25 mol % to 10 mol % poly(ethylene glycol).

Two slides of cultured cells were prepared, using either SK-BR-3 human breast cancer cells or MCF-7 human breast cancer cells. Based on conventional immunohistochemical staining and other data, SK-BR-3 cells have a low score (0) for HER2/ErbB2 expression, while MCF-7 have a high score (3) (see, e.g., refs, infra.). The slides were stained with the red-fluorescing QD-liposome-HER2 antibody conjugates using the protocol as in Example 1, including the sonication step (9). In this case, sonication was performed at 40 kHz for 5 minutes at room temperature. Cell nuclei were counter-stained blue using DAPI, MCF-7 cells were minimally labeled with the labeling reagent, with only a few red-fluorescing C)Ds being visible on the field. In contrast, most of the SK-BR-3 human breast cancer cells were intensely labeled with the labeling reagent.

### Example 3

### Use of Sonication to Reduce Background

A labeling experiment was performed using MCF-7 human breast carcinoma cell buttons having a HER2 score of 1+ (i.e., low HER2-expressing), based on conventional immunohistochemical staining. The same nanoparticle labeling reagent was used as in Example 1. It was observed that without sonication, non-specific binding of the nanoparticle labeling reagent labeled many cells in the field, tending to decorate the perimeter of most cells and clusters of cells. Sonication effectively removed these non-specific bound labeling reagents, leaving only those specifically-bound to HER2 receptors.

### Example 4

### Exemplary Sonicating Device

An exemplary sonicating device consists of an ultrasound generating transducer and microfluidic channels that direct the flow of appropriate buffers and reagent to cover tissues or other biological samples being analyzed. Ultrasound waves may be generated by materials exhibiting the “converse piezoelectric effect”, i.e., stress and strain generated upon application of an electric field. The frequency of the ultrasound waves can be controlled and tuned to suit a particular biological sample or targeting molecule/target (i.e., binding pair) by varying the oscillation of the electric field and. Examples of materials that exhibit the converse piezoelectric effect include but are not limited to gallium orthophosphate (GaPO₄), langasite (La₃Ga₅SiO₁₄), barium titanate (BaTiO₃), lead titanate (PbTiO₃), lead zirconate titanate (PZT), potassium niobate (KNbO₃), niobate (LiNbO₃), tantalate (LiTaO₃), sodium tungstate (NaxWO₃), Ba₂NaNb₅O₅, and Pb₂kNb₅O₁₅. Such piezoelectric materials may be deposited as parallel lines or corrals by conventional thin film methods. The thickness of the deposited material may be about 1 μm, with a width of about 2-10 μm. The device itself may have a dimensions of about 25×75 mm for accommodating standard microscope slides.

### Example 5

### Dynamic Light Scattering of a Nanoparticle Labeling Reagent

Dynamic light scattering (DLS) is a widely used technique for nano/micro-particle sizing and characterization based on the relationship between light scattering and Brownian motion of particles in media. DLS data is analyzed to yield the size (hydrodynamic diameter) of the particles and its distribution. Typical size and size distribution of a nanoparticle labeling reagent is typically between 50 to 400 nm (diameter) and a distribution of 30 to 100 nm centering the main population.

1. A method for labeling a biological target, comprising:
providing a biological target for labeling; and
incubating the biological target with a single-reagent labeling composition comprising
(i) a nanoparticle core structure
(ii) a targeting moleculing specific for the biologic target, and
(iii) at least one luminescent component;
wherein said incubating achieves labeling of the biological target with the single-reagent labeling composition.

2. The method of claim 1, wherein the target molecule is attached to the nanoparticle and the luminescent component is attached to the nanoparticle.

3. The method of claim 1, wherein the target molecule is attached to the nanoparticle and the luminescent component is attached to the targeting molecule.

4. The method of claim 1, wherein the luminescent component is attached to the nanoparticle and the targeting molecule is attached to the luminescent component.

5. The method of claim 1, wherein the nanoparticle core structure is selected from the group consisting of a dendrimer, a hyperbranched polymer, a liposome, a metal oxide, a silicon oxide (silica), a lipid micelle, a lentivirus, a plastic bead, and a polymer oxide.

6. The method of claim 1, wherein the luminescent component is a fluorescent nanocrystal.

7. The method of claim 6, wherein the fluorescent nanocrystal is a quantum dot, a quantum rod, or a quantum wire.

8. The method of claim 1, wherein the targeting molecule is an antibody or a fragment of an antibody having binding specificity to the biological target.

9. The method of claim 1, wherein the targeting molecule is a nucleic acid, a receptor, a ligand, or a structure on a cell.

10. The method of claim 1, further comprising sonicating the biological target following incubation with the labeling composition to remove non-specifically-bound labeling composition.

11. A composition for labeling a biological target comprising:
a single labeling reagent comprising a nanoparticle core structure having a plurality of binding sites for multiplex attachment of at least one targeting molecule, and at least one luminescent component having one or more preselected wavelengths.

12. The composition of claim 11, wherein the nanoparticle core structure is selected from the group consisting of a dendrimer, a liposome, a metal oxide, a silicon oxide (silica), a lipid micelle, a lentivirus, a plastic bead, and a polymer micelle.

13. The composition of claim 11, wherein the luminescent component is a fluorescent nanocrystal.

14. The composition of claim 13, wherein the fluorescent nanocrystal is a quantum dot.

15. The composition of claim 14, wherein the fluorescent nanocrystal is modified with carboxyl groups to facilitate attachment to the nanoparticle core structure.

16. The composition of claim 11, wherein the at least one targeting molecule is an antibody or a fragment of an antibody having binding specificity to the biological target.

17. The composition of claim 16, wherein the antibody is specific for HER-2.

18. The composition of claim 11, wherein the targeting molecule is a nucleic acid, a receptor or a ligand.

19. A composition for labeling a biological target, comprising:
a single labeling regent comprising a nanoparticle core structure, at least one targeting molecule, and at least one luminescent component having one or more preselected wavelengths.

20. The composition of claim 19, wherein the targeting molecule is attached to the nanoparticle and the luminescent component is attached to the nanoparticle.

21. The composition of claim 19, wherein the targeting molecule is attached to the nanoparticle and the luminescent component is attached to the targeting molecule.

22. The composition of claim 19, wherein the luminescent component is attached to the nanoparticle and the targeting molecule is attached to the luminescent component.

23. A method for reducing non-specific labeling of a biological sample using a composition according to claim 11, comprising,
sonicating the biological material following binding of the nanoparticle labeling reagent to remove non-specifically bound nanoparticle labeling reagent.

24. A method for detecting a biological target, comprising contacting the biological target with a composition according to claim 11.
